(19) United States
(12) Patent Application Publication
(10) Pub. No.: US 2018/0199981 A1
(43) Pub. Date: Jul. 19, 2018

(54) SYSTEMS AND METHODS TO PREVENT OR SIGNIFICANTLY INHIBIT GAS PROGRESSION DURING SPRAY CRYOTHERAPY

(71) Applicant: CSA Medical, Inc., Lexington, MA (US)

(72) Inventors: Ellen E. Sheets, Boston, MA (US); Brian M. Hanley, Reading, MA (US); John P. O'Connor, Andover, MA (US)

(21) Appl. No.: 15/874,414

(22) Filed: Jan. 18, 2018

Related U.S. Application Data
(60) Provisional application No. 62/448,196, filed on Jan. 19, 2017.

(51) Int. Cl. A61B 18/02; A61B 1/00; A61B 1/018

(52) U.S. Cl. CPC A61B 18/0218; A61B 2017/00296; A61B 1/018; A61B 1/00087

(57) ABSTRACT

The present disclosure relates generally to the field of cryotherapy. In particular, the present disclosure relates to cryotherapy catheters and systems that utilize a detachable expandable member to prevent or significantly inhibit cryogen gases from accumulating and progressing distally beyond a specific region within a body lumen.

### PRIORITY

This application claims the benefit of priority under 35 U.S.C. § 119 to U.S. Provisional Patent Application Ser. No. 62/448,196, filed Jan. 19, 2017, which is incorporated by reference herein in its entirety and for all purposes.

### FIELD

The present disclosure relates generally to the field of cryotherapy. In particular, the present disclosure relates to cryotherapy catheters and systems, etc. that utilize a detachable expandable member to prevent or significantly inhibit cryogen gases from accumulating and progressing distally beyond a specific region within a body lumen.

### BACKGROUND

As an example of cryotherapy, cryoablation is a surgical procedure in which diseased, damaged or otherwise undesirable tissue (collectively referred to herein as “target tissue”) is destroyed by focal delivery of a cryogen spray under pressure. These systems along with other cryotherapy systems are typically referred to as cryoablation systems, cryospray systems, cryospray ablation systems, cryosurgery systems, cryosurgery spray systems and/or cryogen spray ablation systems. As typically used, “cryogen” refers to any fluid (e.g., gas, liquefied gas or other fluid known to one of ordinary skill in the art) with a sufficiently low boiling point (i.e., below approximately −153° C.) for therapeutically effective use during a cryogenic surgical procedure. Suitable cryogens may include, for example, liquid argon, liquid nitrogen and liquid helium. Pseudo-cryogens such as liquid carbon dioxide and liquid nitrous oxide that have a boiling temperature above −153° C. but still very low (e.g., −89° C. for liquid N₂O) may also be used.

For example, during operation of a cryospray ablation system, a medical professional (e.g., clinician, technician, physician, surgeon, etc.) directs a cryogen spray onto the surface of a treatment area via a cryogen delivery catheter. The medical professional may target the cryogen spray visually through a video-assisted device or scope, such as a bronchoscope, endoscope, colonoscope or ureteroscope. Cryogen spray exits the cryogen delivery catheter at a temperature ranging from 0° C. to −196° C., causing the target tissue to freeze or “cryofrost.” As liquid cryogen exits the cryogen delivery catheter and impacts upon the target, it converts to a gaseous state with a significant increase in volume. For example, 1 cubic centimeter (cm³) of liquid nitrogen converts to 694 cm³ of nitrogen gas at body temperature. If not properly vented from the patient and/or allowed to progress further into the body from the treatment site, these expanding gases cause undue distention and may have life-threatening consequences, including, for example, pneumothorax of the lungs and perforations of the upper or lower gastrointestinal (GI) tract.

Accordingly, various advantages may be realized by cryotherapy systems and methods as disclosed herein which prevent or significantly inhibit cryogen gases from accumulating and progressing distally beyond a specific region within body lumens and allow for adequate ventilation of the gases, without obstructing the cryogen gases from contacting the treatment area.

### SUMMARY

In one aspect, the present disclosure provides a system, comprising a delivery device which includes a distal end, a proximal end, and a lumen extending therebetween; an expandable member which includes an outer surface, and an inner surface defining an interior region; and a valve disposed on the outer surface of the expandable member, and in fluid communication with the interior region, wherein the distal end of the delivery device is reversibly attachable to the valve such that the interior region of the expandable member is in fluid communication with the lumen of the delivery device via the valve. The distal end of the delivery device may include an attachment element configured to engage a corresponding attachment element of the valve. The distal end of the delivery device may include a male attachment element configured to engage a corresponding female attachment element of the valve. The corresponding male and female attachment elements may include, by way of non-limiting example, threaded surfaces or luer lock surfaces. A portion of the male attachment element may be configured to expand within a portion of the female attachment element. The valve may be seated within a housing. The housing may further include a funnel member configured to guide the distal end of the delivery device into the housing.

In another aspect, the present disclosure provides a system, comprising a delivery device which includes a distal end, a proximal end and a lumen extending therebetween; an expandable member defining an interior region; and a valve disposed between and having a fluid communication to the lumen and the interior region, wherein the valve and expandable member are reversibly detachable from the distal end of the delivery device. The valve may include, by way of non-limiting example, a female attachment element configured to engage a corresponding male attachment element of the delivery device. The male attachment element may radially expand when inserted a predetermined depth into the female attachment element. The system may further include a support member extending along a longitudinal axis of the expandable member within the interior region. The expandable member may include a balloon formed from a non-compliant or semi-compliant polymer material, including, by way of non-limiting example, PEBAX, PET, PEN, PBT, PEEK, Hytrel, polyurethane and nylon. The system may further include an endoscope which includes a detachable external lumen or rail system. The delivery device may be configured to be inserted within the detachable external lumen or rail system, such that the delivery device is slidable along the endoscope. The system may also include one or more fastening elements configured to slidably attach to an outer surface of an endoscope. Alternatively, the system may be slidably disposed within a working channel of an endoscope.

In another aspect, the present disclosure provides a method, which includes positioning a delivery device that includes a detachable expandable member at a target location within a body lumen; inflating the detachable expandable member to contact opposing walls of the body lumen at the target location; detaching the detachable expandable member from the delivery device; and performing a procedure in the body lumen at a position proximal to the target location. The method may further include re-attaching the delivery device to the detachable expandable member. The method may further include deflating the detachable expandable member. The method may further include removing the delivery device and detachable expandable member from the body lumen. The procedure may include, by way of non-limiting example, a cryotherapy procedure. The body lumen may include an esophagus, trachea, lung, colon, large intestine or stomach.

### BRIEF DESCRIPTION OF THE DRAWINGS

Non-limiting embodiments of the present disclosure are described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment shown where illustration is not necessary to allow those of ordinary skill in the art to understand the disclosure. In the figures:

FIGS. 1A-1C provide perspective views of a detachable expandable member, according to an embodiment of the present disclosure.

FIGS. 2A-2C provide perspective views of a detachable expandable member, according to another embodiment of the present disclosure.

FIGS. 3A-3C provide perspective views of valve configurations, according to further embodiments of the present disclosure. FIG. 3D provides a perspective view of a funnel member for various valve configurations, according to an embodiment of the present disclosure.

FIGS. 4A-4C illustrate a cryotherapy procedure with a detachable expandable member performed within the lower gastrointestinal tract, according to one embodiment of the present disclosure.

FIGS. 5A-5C illustrate a cryotherapy procedure with a detachable expandable member performed within the upper gastrointestinal tract, according to another embodiment of the present disclosure.

FIGS. 6A-6C illustrate a cryotherapy procedure with a detachable expandable member performed within the upper gastrointestinal tract, according to another embodiment of the present disclosure.

FIGS. 7A-7B illustrate a cryotherapy procedure with a detachable expandable member performed within the respiratory tract, according to another embodiment of the present disclosure.

FIGS. 8A-8G illustrate representative steps involved in delivering and retrieving a detachable expandable member, according to another embodiment of the present disclosure.

### DETAILED DESCRIPTION

The present disclosure is not limited to the particular embodiments described. The terminology used herein is for the purpose of describing particular embodiments only, and is not intended to be limiting beyond the scope of the appended claims. Unless otherwise defined, all technical terms used herein have the same meaning as commonly understood by one of ordinary skill in the art to which the disclosure belongs.

Although embodiments of the present disclosure are described with specific reference to cryotherapy systems for use within the upper and lower GI tracts and respiratory system, the various systems and methods may be used in a variety of other body passageways, organs and/or cavities, such as the vascular system, urogenital system, lymphatic system, neurological system and the like. The various embodiments of the present disclosure are not necessarily limited to cryotherapy procedures, but may be employed in other medical procedures in which it is desirable to employ a detachable expandable backstop to prevent or significantly inhibit the progress of a substance or medical instrument further into a body passage.

As used herein, the singular forms “a,” “an,” and “the” are intended to include the plural forms as well, unless the context clearly indicates otherwise. It will be further understood that the terms “comprises” and/or “comprising,” or “includes” and/or “including” when used herein, specify the presence of stated features, regions, steps elements and/or components, but do not preclude the presence or addition of one or more other features, regions, integers, steps, operations, elements, components and/or groups thereof.

As used herein, the term “distal” refers to the end farthest away from the medical professional when introducing a device into a patient, while the term “proximal” refers to the end closest to the medical professional when introducing a device into a patient.

As used herein, the term “expandable” refers to the ability to self-expand or cause to be expanded in diameter from a “collapsed,” “unexpanded” or “deflated” configuration to an “expanded” or “inflated” configuration. As used herein, “diameter” refers to the distance of a straight line extending between two points and does not necessarily indicate a particular shape.

As used herein, the term “passive venting” refers to the unassisted egress of gases from within a body lumen to an external location, through a body lumen and natural orifice or through a ventilation tube passing through the same. As used herein, the term “active venting” refers to the mechanically-assisted egress (e.g., via a suction source) of gases from with a body lumen to an external location (e.g., through a ventilation tube, through an endoscope working channel, or through a working channel of a cryogen delivery catheter or other catheter).

As used herein, the term “retroflex” refers to the ability of a medical instrument to bend or turn approximately 180° about a radius of curvature.

The present disclosure generally provides cryotherapy systems configured to prevent or significantly inhibit the accumulation and distal progression of materials and/or substances, including, but not limited to, cryospray gases (hereafter referred to as “cryospray”), within a body lumen, without obstructing the cryospray from contacting the entire surface of a treatment area. Exemplary cryotherapy systems in which the present disclosure may be implemented include, but are not limited to, those systems described commonly owned U.S. Pat. Nos. 9,301,796, 9,144,449, 7,225,693, 7,025,672, 6,383,181 and 6,027,499 and U.S. patent application Ser. Nos. 11/956,890, 12/022,013, 14/012,320 and 14/869,814, each of which are herein incorporated by reference in their entirety.

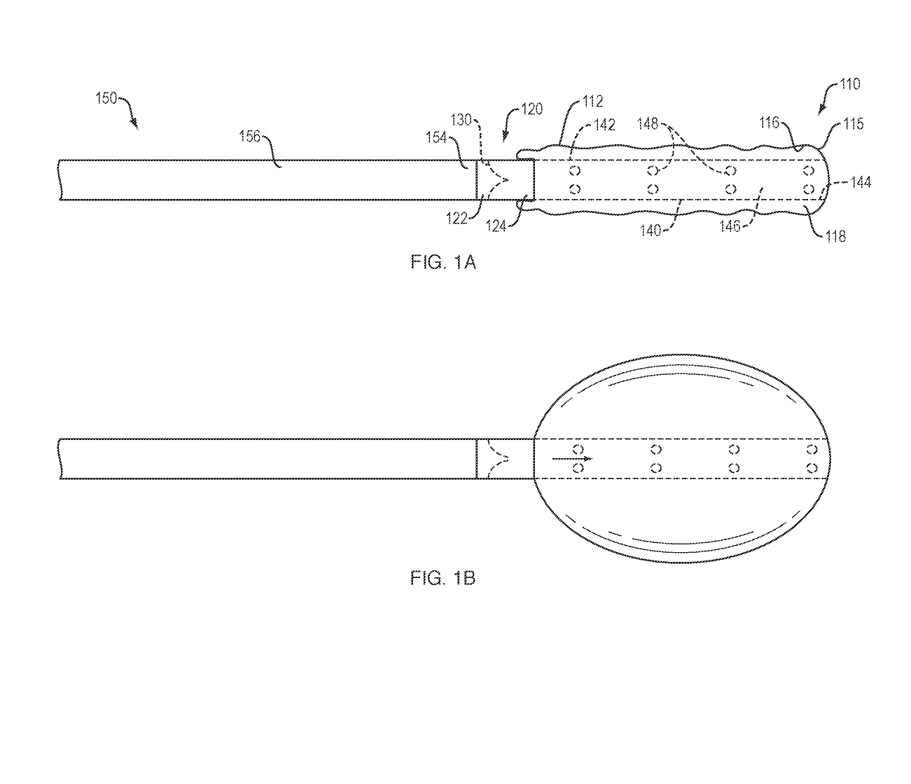

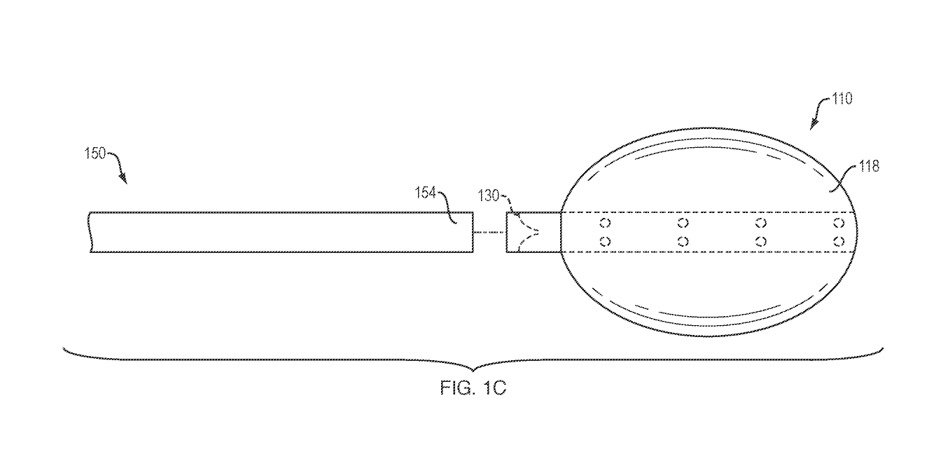

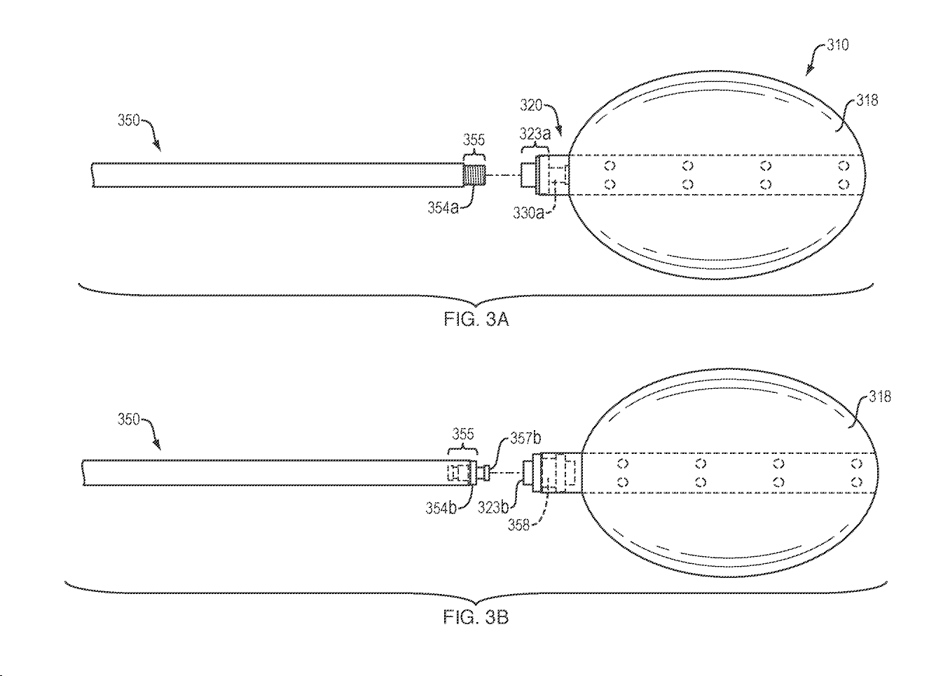

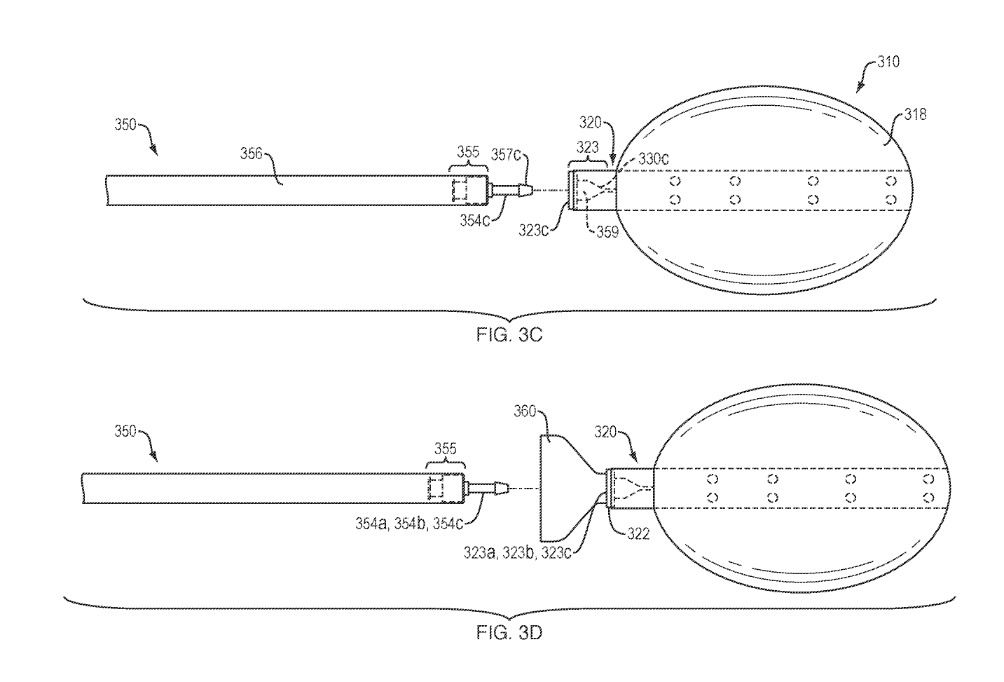

Referring to FIG. 1A, in one embodiment, the present disclosure provides a detachable expandable member 110 (e.g., balloon, etc.) reversibly attached to a delivery device 150 (e.g., catheter, probe, tether, insertion device, etc.). The expandable member 110 may include an outer surface 115, and an inner surface 116 defining an interior region 118. The delivery device 150 may include a proximal end (not shown), a distal end 154 and a lumen 156 extending therebetween. A housing 120 comprising a proximal end 122 and a distal end 124 may be attached to a proximal end 112 of the expandable member 110, such that a valve 130 seated within the housing 120 is disposed between and in fluid communication with the lumen 156 of the delivery device 150 and the interior region 118 of the expandable member 110, when the expandable member is attached to the delivery device. A proximal portion of the housing 120 may include an attachment or connection element (e.g., female connector, receiving member, mating portion, catcher, etc.) configured to receive and reversibly engage a corresponding attachment or connection element (e.g., male connector, mating portion, etc.) on a distal portion of the delivery device (See, e.g., FIGS. 3A-3D).

The expandable member 110 may further include a support member 140 extending along a longitudinal axis of the expandable member 110 within the interior region 118. The support member 140 may provide or assist with the requisite pushability and steerability for placement of the expandable member within a body lumen. The support member 140 may include a proximal end 142, a distal end 144 and a lumen 146 extending at least partially therebetween. The proximal end 142 of support member 140 may be connected (e.g., bonded, affixed, attached, integrally formed with, etc.) to the distal end 124 of the housing 120, and the distal end 144 of support member 140 may be attached to a portion of the inner and/or outer surface 116, 115 of the expandable member 110. The support member 140 may further include one or more inflation/deflation ports 148 configured to place the lumen 146 in fluid communication with the interior region 118 of the expandable member 110. Although the inflation/deflation ports 148 are depicted as circular and evenly spaced along the length of the support member 140, in various embodiments, the number, shape, location and/or orientation of the inflation/deflation ports along the support member may vary. For example, the inflation/deflation ports may be preferentially positioned at the proximal or distal ends 142, 144 of the support member 140. Alternatively, the inflation/deflation ports 148 may be positioned at opposite ends of the expandable member 110.

The expandable member 110 may form a fluid-tight seal around the respective distal ends 144, 124 of the support member 140 and housing 120, such that the expandable member 110 may move between the unexpanded (FIG. 1A) and expanded (FIG. 1B) configurations as inflation fluid (e.g., gas, liquid, etc.) is delivered into, or removed from, the interior region 118. For example, inflation/deflation ports 148 may allow inflation fluid to be delivered under pressure from an external fluid source through the lumen 156 and valve 130 into the interior region 118, in order to move the expandable member 110 from an unexpanded (e.g., deflated, collapsed) configuration to an expanded (e.g., inflated) configuration. The inflation/deflation ports 148 may likewise allow inflation fluid to be removed (e.g., under suction or vacuum from the same or different external source) from the interior region 118 through the valve 130 and lumen 156 to deflate the expandable member from the expanded to unexpanded configuration. The inflation fluid may include a variety of physiologically inert liquids (e.g., buffered solutions such as sterile saline) or gases (e.g., air, oxygen, nitrogen, hydrogen, carbon dioxide, helium, etc.) as are known in the art.

Flow of inflation fluid between the external fluid source and interior region 118 of the expandable member 110 may be performed manually using, e.g., a syringe, or automatically using an external system. The syringe (or external system) may include a pressure gauge configured to allow a medical professional to confirm that the expandable member 110 is sufficiently inflated to contact opposing walls of a body lumen without over-expansion, in order to prevent or significantly inhibit the distal progression of cryospray, and/or sufficiently deflated for safe removal from (or repositioning within) the body lumen. For example, an automatically operated external system may include a pressure sensor configured to prevent the delivery of cryogen if the expandable member 110 is either deflated or insufficiently inflated to establish proper contact with the tissue walls of the body lumen. Similarly, the inner or outer surface 116, 115 of the expandable member 110 may include one or more sensors (e.g., pressure sensors, temperature sensors, etc.) to allow the temperature and/or pressure of the expandable member to be monitored throughout the cryotherapy procedure. For example, one or more pressure sensors on an inner surface 116 of the expandable member 110 may allow the medical professional to introduce or remove inflation fluid until a desired level of inflation (e.g., internal pressure) is achieved. In addition, or alternatively, one or more pressure sensors on an outer surface 115 of the expandable member 110 may allow the medical professional to monitor the pressure exerted by the expandable member against opposing walls of the body lumen (e.g., external pressure). The medical professional may adjust (e.g., increase or decrease) the inflation/deflation level as necessary to maintain desired contact between the expandable member and body lumen without causing trauma to the body lumen and patient. In one embodiment, the sensors may be configured to wirelessly transmit the pressure and/or temperature measurements such that the medical professional may monitor the inflation/deflated level of the expandable member, including when detached from the delivery device during a cryotherapy procedure. For example, if the pressure within the expandable member 110 decreases below a threshold level during the cryotherapy procedure (e.g., due to leakage of the inflation fluid, or condensation of the inflation fluid due to proximity to the cryospray), the medical professional may stop the cryotherapy procedure and reposition or re-inflate the expandable member to prevent harm to the patient.

Referring to FIG. 1C, when the expandable member 110 is released (e.g., disconnected, detached, etc.) from the distal end 154 of the delivery device 150, the valve 130 prevents the inflation fluid from exiting the interior region 118 thereby maintaining the expandable member 110 in the expanded configuration.

The support member 140 and/or delivery device 150 may include appropriate sizes, dimensions and/or suitable materials to facilitate navigation, e.g., within narrow tortuous body passages or within a working channel of an endoscope, depending on the demands of the particular application. The expandable member 110 may also be provided in a variety of different expanded dimensions in order to prevent or significantly inhibit progression of gases in a range of body lumen sizes. In various embodiments, the expandable member, e.g., as a balloon, may include a combination of polymeric and semi-compliant materials, such as thermoplastics and/or thermosets. The semi-compliant nature of these materials may be desirable in some embodiments to ensure that the expandable member better conforms to the shape of the body lumen in which it is placed, but does not over-expand within the target body lumen.

Examples of thermoplastics include polyolefins; polyamides (e.g., nylon, such as nylon 12, nylon 11, nylon 6/12, nylon 6, nylon 66); polyesters (e.g., polyethylene terephthalate (PET), polybutylene terephthalate (PBT), polyethylene naphthalate (PEN), polytrimethylene terephthalate (PTT)); polyethers; polyurethanes; polyvinyls; polyacrylics; fluoropolymers; copolymers and block copolymers thereof, such as block copolymers of polyether and polyamide (e.g., PEBAX®); and mixtures thereof. Examples of thermosets include elastomers (e.g., EPDM), epichlorohydrin, polyureas, nitrile butadiene elastomers and silicones. Other examples of thermosets include epoxies and isocyanates. Biocompatible thermosets may also be used. Biocompatible thermosets include, for example, biodegradable polycaprolactone, poly(dimethylsiloxane) containing polyurethanes and ureas and polysiloxanes. Ultraviolet curable polymers, such as polyimides and acrylic or methacrylic polymers and copolymers can also be used. Other examples of polymers that can be used in expandable members include polyethylenes, polyethylene ionomers, polyethylene copolymers, polyetheretherketone (PEEK), thermoplastic polyester elastomers (e.g., Hytrel®) and combinations thereof. Other polymers are described, for example, in U.S. Pat. Pub. No. 2005/0043679, filed on Aug. 21, 2003, entitled “Medical Balloons,” the disclosure of which is incorporated in its entirety herein by reference.

In another embodiment, a compliant expandable member may be desirable to establish and maintain firm contact with the tissue wall of amorphous and/or asymmetrically shaped lumens. As compared to semi-compliant materials, an expandable member formed from a compliant material will expand indefinitely (i.e., does not have a fixed final diameter). These expandable members are composed of materials with compliances preferably in the range of 10% to 800%, and more preferably in the range of 50% to 200%. Examples of compliant materials include elastomers such as silicone rubber, ethylene-propylene-diene copolymers, butyl rubber, styrene-isobutylene-styrene copolymers, urethanes, and latexes, among others.

In the various embodiments described here and otherwise, expandable members may be folded, pleated and/or covered by a sheath until deployed to protect the expandable member and facilitate delivery within/through body lumens. Radiopaque materials may be incorporated into or onto the compliant or semi-compliant materials, or on the distal and proximal ends of the support member, or on the valve housing and distal end of the delivery device, or on any combination of the above, to allow the location of the expandable member to be visualized with systems capable of detection of radiopaque materials within the patient, such as fluoroscopy imaging.

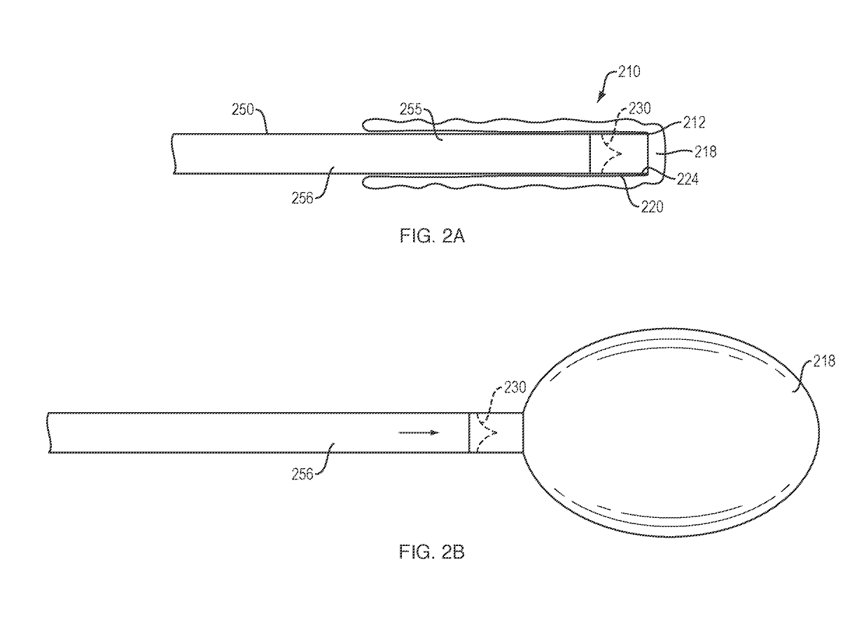

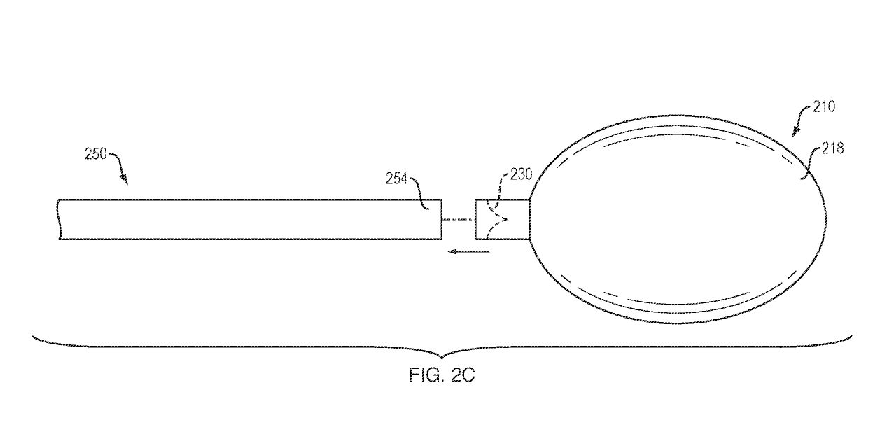

Referring to FIG. 2A, in one embodiment, a proximal end 212 of the expandable member 210 may form a fluid-tight seal around the distal end 224 of the housing 220 such that that expandable member 210 folds back along the outer surface of the housing 220 and a distal portion 255 of the delivery device 250. The distal portion 255 of the delivery device 250 may impart the requisite steerability and pushability for placement of the expandable member 210 within the body lumen. The expandable member 210 may be inflated and deflated between unexpanded (FIG. 2A) and expanded (FIG. 2B) configurations as inflation fluid is delivered into, or removed from, the interior region 218. For example, inflation fluid may be delivered under pressure from an external fluid source through the lumen 256 and valve 230 into the interior region 218 to move the expandable member 210 from an unexpanded (e.g., deflated, collapsed) configuration (FIG. 2A) to an expanded (e.g., inflated) configuration (FIG. 2B). Inflation fluid may likewise be returned (e.g., removed) under suction from the interior region 218 through the valve 230 and lumen 256 to the external fluid source to move the expandable member from the expanded to unexpanded configuration, such as described above with respect to the embodiment of FIGS. 1A-1C. In this and the above and other embodiments, in order to avoid damage to the body lumen wall, the expandable member may be configured to unfold or unroll in a longitudinal direction relative to the delivery device prior to moving outward from the unexpanded to expanded configuration. Referring to FIG. 2C, when the expandable member 210 is released (e.g., disconnected, detached, etc.) from the distal end 254 of the delivery device 250, the valve 230 prevents the inflation fluid from exiting the interior region 218 thereby maintaining the expandable member 210 in the expanded configuration. For example, the valve 230 may include one or more movable elements configured to be overcome by a threshold pressure (e.g., fluid flow) in one direction such that the valve opens for inflation purposes, but which are not overcome by the same (or greater) threshold pressure in the opposite direction to maintain the expandable member in the inflated configuration. For deflation purposes, it may be necessary to breach or open the valve by introducing, e.g., a male attachment element or stylet through the lumen of the delivery device. The valve 130, 230 depicted in FIGS. 1A-1C and 2A-2C may be connected to the delivery device using, e.g., one or both of the connection configurations of FIG. 3 (as described below).

Referring to FIGS. 3A-3D, various other configurations of valve connections for use with expandable members of the present disclosure are described, utilizing an expandable member with a support member as an example. As shown, the distal portion 355 of the delivery device 350 may be configured to form a reversibly detachable connection with the valve in proximal portion 323 of the housing 320. For example, in one embodiment (FIG. 3A), the distal portion 355 of the delivery device 350 may include a threaded male attachment element 354a configured to engage a corresponding threaded female attachment element 323a on an inner surface of the proximal portion 323 of the housing 320 (FIG. 3A). With the expandable member securely positioned within a body lumen in the expanded configuration, the medical professional may detach the expandable member 310 from the delivery device 350 by unthreading the male attachment element from the female attachment element. Similarly, the medical professional may re-attach the delivery device 350 to the expandable member 310 by re-threading the male attachment element into the female attachment element. Alternatively, the reversibly detachable valve connection may be a luer lock configuration (FIG. 3B), wherein the male attachment element (354b) is attached to the female attachment element (323b) of the luer lock configuration using a “push and twist” (i.e., clockwise rotation) motion. With the expandable member securely positioned within a body lumen and inflated in the expanded configuration, the medical professional may detach the expandable member from the delivery device by disconnecting the male attachment element from the female attachment element using a “twist and pull” motion (e.g., counter-clockwise rotation). In another alternative, the reversibly detachable valve connection may be an expandable valve post and valve seat arrangement (FIG. 3C), wherein a valve post 357c of attachment element 354c is advanced into the corresponding attachment element 323c, and a portion of the valve post 357c may be expanded to secure attachment element 354c within attachment element 323c.

In the valve configuration of FIG. 3A, when the respective attachment elements 354a, 323a of the delivery device 350 and housing 320 are fully engaged (e.g., connected, attached, interlocked, etc.), the valve 330a seated within the housing 320 may move or may be caused to be move to an open configuration such that the expandable member 310 may inflate and deflate between the unexpanded and expanded configurations as inflation fluid is delivered into, or removed from, the interior region 318. Similarly, when the respective attachment elements 354a, 323a of the delivery device 350 and housing 320 are disengaged (e.g., disconnected, detached, etc.) the valve 330a may move to a closed configuration to prevent the inflation fluid from exiting the interior region 318, thereby maintaining the expandable member 310 in the expanded configuration. For example, the engagement forces between the corresponding attachment elements of the delivery device and housing may exert outward forces within the housing such that the valve moves to an open configuration, and conversely, when those engagement forces are released/removed the valve may return to the closed configuration.

In another embodiment (FIG. 3B), the distal portion 355 of the delivery device 350 may include male attachment element 354b configured to engage a corresponding female attachment element 323b seated within the housing 320 (FIG. 3B). When the respective attachment elements 354b, 323b of the delivery device 350 and housing 320 are fully engaged, a valve post 357b of the attachment element 354b engages a valve piston 358 within the attachment element 323b and moves to an open configuration such that the expandable member 310 may inflate and deflate between unexpanded and expanded configurations as inflation fluid is delivered into, or removed from, the interior region 318. Similarly, when the respective attachment elements 354b, 323b of the delivery device 350 and housing 320 are disengaged, the valve piston 358 may return to a closed configuration to prevent the inflation fluid from exiting the interior region 318, thereby maintaining the expandable member in the expanded configuration.

In another embodiment of a valve configuration depicted in FIG. 3C, the distal portion 355 of the delivery device 350 may include an attachment element 354c configured to engage a corresponding attachment element 323c on the proximal portion 323 of the housing 320 such that a valve post 357c passes through (e.g., penetrates) the valve sleeve 359 (e.g., duckbill valve, etc.) seated within the housing 320 (FIG. 3C). When the respective attachment elements 354c, 323c of the delivery device 350 and housing 320 are fully engaged, the valve sleeve 359 seated within the housing 320 with the valve post 357c inserted therethrough, moves to an open configuration such that fluid may pass through the hollow lumen of valve post 357c, and the expandable member 310 may inflate and deflate between unexpanded and expanded configurations as inflation fluid is delivered into, or removed from, the interior region 318. Similarly, when the respective attachment elements 354c, 323c of the delivery device 350 and housing 320 are disengaged and valve post 357c is removed from the valve sleeve 359, valve sleeve 359 may move or collapse to a closed configuration to prevent the inflation fluid from exiting the interior region 318, thereby maintaining the expandable member in the expanded configuration.

Alternatively, the reversibly detachable valve connection may include an insert/expand configuration, in which the male attachment element (354c) includes a distal component that is inserted into the female attachment element (323c) of the detachable unit. For example, when the valve post 357c of attachment element 354c is advanced a predetermined depth into the corresponding attachment element 323c, a portion of the valve post 357c may expand to secure attachment element 354c within attachment element 323c. This secure interaction may be overcome by applying sufficient force (e.g., retracting, withdrawing, etc.) to the attachment element 354c that the expanded portion of the valve post 357c is pulled out of the attachment element 323c or the expanded portion of the valve post 357c returns to a non-expanded configuration for removal from the attachment element 323c.

In addition, or alternatively, inflation fluid may be delivered into the interior region 318 through the valve 330c in the absence of valve post 357c, provided that valve sleeve 359 has a threshold opening pressure that is overcome by the inflation fluid. For example, the inflation fluid may be delivered through the lumen 356 of the delivery device 350 with sufficient pressure to force the opposing walls of the valve sleeve 359 to open. When the flow of inflation fluid stops, the valve 330c may return to a closed configuration to maintain the expandable member in the expanded configuration. When the expandable member is reattached, a stylet may be passed through the delivery device and valve sleeve 359 in order to open the valve and allow the inflation fluid to exit the through the valve sleeve around the stylet.

Referring to FIG. 3D, in one embodiment, an open-ended guide member 360 (e.g., funnel member, basket, etc.) may be attached to the proximal end 322 of the housing 320 to help guide the respective attachment elements 354a, 354b, 354c of the delivery device 350 into corresponding attachment elements 323a, 323b, 323c of the housing 320. In one embodiment, the distal portion 355 of the delivery device 350 may also include a light source and camera to allow the medical professional to visualize the corresponding attachment elements on the delivery device 350 and housing 320 to ensure proper alignment as the delivery device and expandable member are reconnected. Each of the connection embodiments disclosed herein (e.g., threads, luer lock, etc.) may be interchangeable with any of the expandable member configurations, and any of the valve configurations (e.g., duckbill valve, sleeve, check valve, etc.) may be interchangeable with any of the various expandable members and any of the connection embodiments.

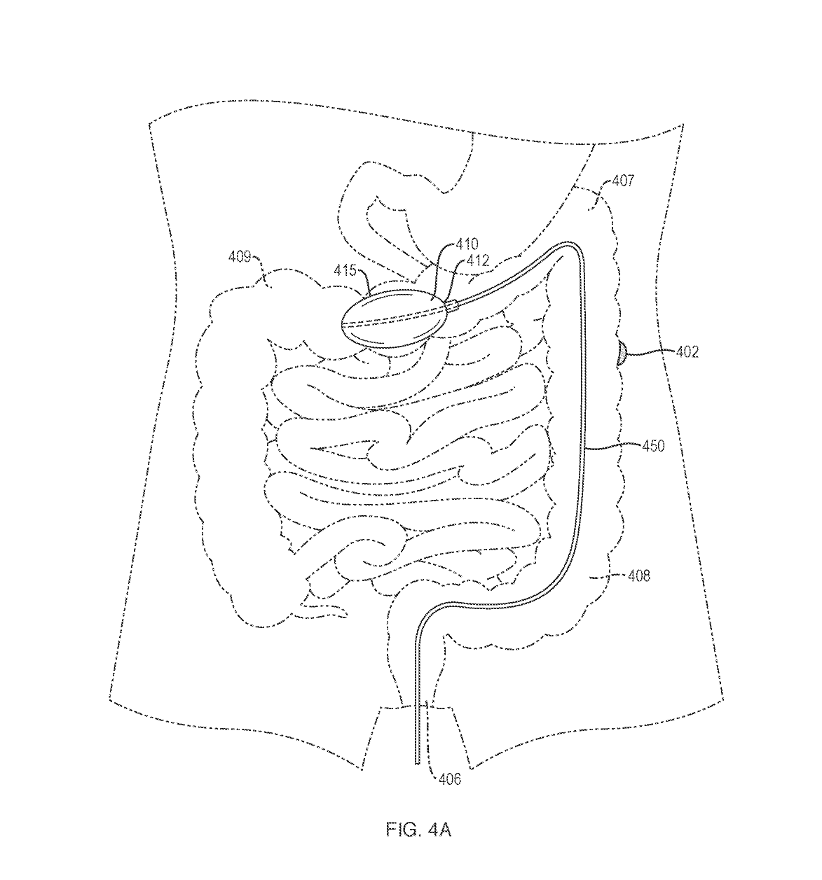

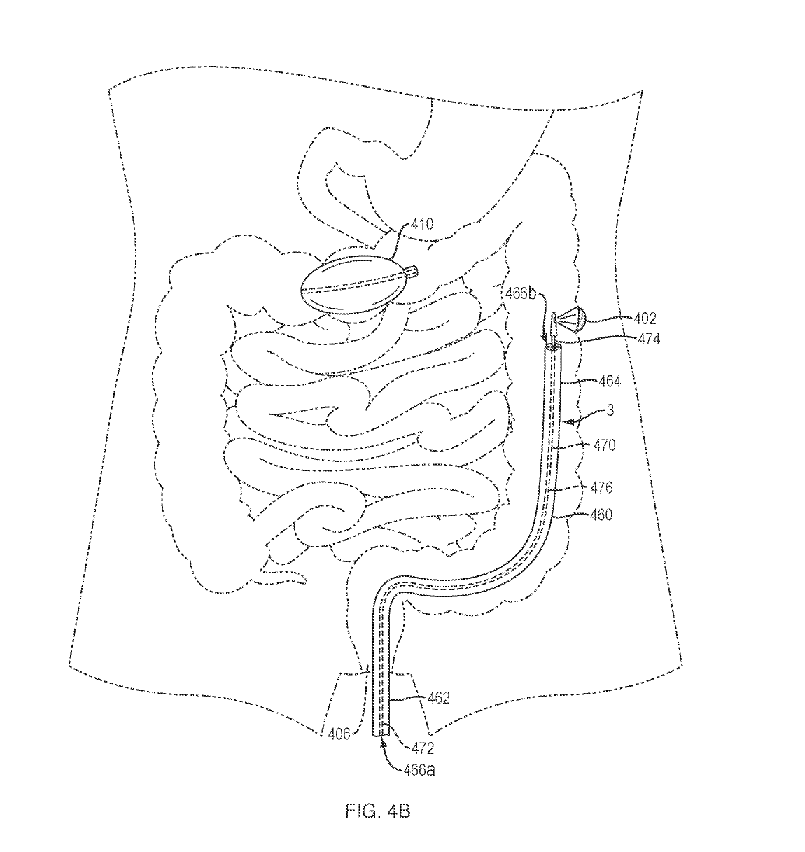

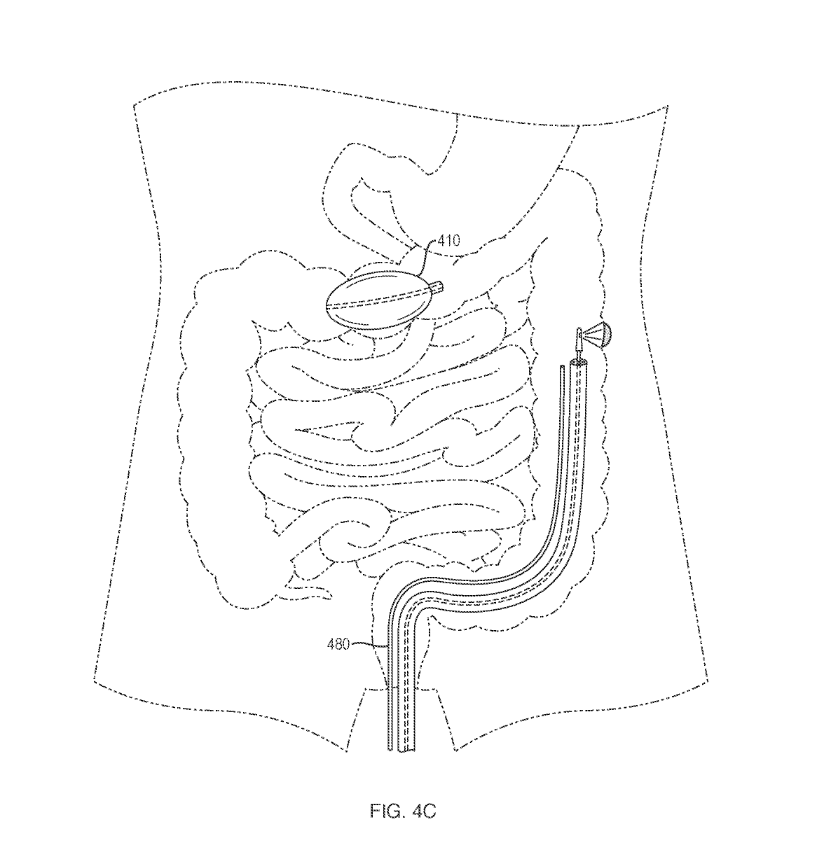

Referring to FIG. 4A, in use, and by way of example, the delivery device 450 and attached expandable member 410 may be introduced in the deflated configuration through the rectum 406 into the colon 408 distally beyond a target tissue 402. The expandable member 410 should be positioned sufficiently distal to the target tissue 402 such that cryospray delivered from the cryogen delivery catheter (FIG. 4B) does not cause the inflation fluid within the expandable member to freeze, or the gases within the expandable member to condense to the point that the expandable member contracts/deflates. Alternatively, inflation fluids resistant to freezing at cryogen temperatures may be chosen so the expandable member can be located closer to the cryogen delivery catheter. Once properly positioned (e.g., between the splenic flexure 407 and hepatic flexure 409, the expandable member 410 is moved to the expanded configuration such that at least a portion of the outer surface 415 contacts all, or substantially all, of the tissues about a circumference of the colon wall. With the expandable member 410 secured in the inflated configuration against the colon wall, the proximal end 412 of the expandable member 410 and housing 420 is disconnected (e.g., detached, released, etc.) from the delivery device 450 and the delivery device 450 may be withdrawn from the patient. Alternatively, rather than completely removing the delivery device 450 device from the patient, the delivery device 450 may be retracted to a position proximal of the target tissue.

Referring to FIG. 4B, after the delivery device has been detached from the expandable member 410 and withdrawn from the patient, a cryotherapy system 3 may be advanced through the rectum 406 and positioned adjacent to the target tissue 402. In one embodiment, the cryotherapy system 3 may include an endoscope 460 comprising a proximal portion 462, a distal portion 464 and a first working channel 466a extending therebetween. The endoscope 460 may include any appropriate size, although smaller diagnostic endoscopes are preferably used to facilitate navigation within body passageways and facilitate patient comfort.

A cryogen delivery catheter 470 may be disposed within the first working channel 466a of the endoscope 460. The cryogen delivery catheter 470 may include a proximal end 472, a distal end 474 and a lumen 476 extending therebetween. The distal end 474 may include closed or open-ended configurations, with or without side apertures disposed around a portion or whole of the circumference thereof. Cryogen (e.g., liquid nitrogen) may be delivered from an external storage tank (not depicted) connected to the proximal end 472 of the cryogen delivery catheter 470, through the lumen 476 to exit through side and/or end aperture(s) at distal end 474. The distal end 474 of the cryogen delivery catheter 470 may include one or more apertures configured to convert the cryogen flowing through the lumen 476 into a pressurized, e.g., low pressure, cryospray. The cryogen warms and boils as it exits the cryogen delivery catheter 470, resulting in a cold cryospray emerging from the distal end 474 onto the target tissue 402. Freezing of the target tissue may be visualized by the acquisition of a white color, referred to as cryofrost. The white color indicates the onset of mucosal tissue freezing to initiate destruction of the target tissue.

The medical professional may increase or decrease the duration of the cryospray treatment depending on the size and/or depth of the target tissue. The cryogen delivery catheter may include various sensors, e.g., temperature sensor, and may be connected to a console with controls that may be necessary or useful to control and monitor a cryospray procedure, including for example regulation of cryogen flow based on temperature feedback, other procedural parameters, venting, etc. In one embodiment, the cryogen delivery catheter may be constructed of three layers of flexible polyimide, surrounded by a stainless steel braid, which is coated with an outer layer of Pebax. As understood by those in the art, extrusion of Pebax over the stainless steel braid allows the Pebax to wick through the pitch of the steel braid, helping to prevent kinking, breaking or delaminating during retroflex of the cryogen delivery catheter.

As apparent to those of skill in the art, the cryogen delivery catheter of the present disclosure may include a variety of suitable materials and/or dimensions depending on the demands of the particular application. The endo scope 460 may further include a second working channel 466b configured to vent the cryospray delivered from the cryogen delivery catheter 470. In one embodiment, the second working channel 466b is configured for passive venting of the cryospray. In another embodiment, the second working channel 466b is connected to a suction source (e.g., pump, not depicted) to facilitate active venting of the cryospray.

Referring to FIG. 4C, in addition, or alternatively, a vent tube 480 (e.g., cryogen decompression tube) may be advanced through the body cavity alongside the endoscope for active or passive venting of the cryospray.

As will be understood by those in the art, the diameter of the second working channel 466b (or vent tube) through which the cryospray passively or actively vents must be adequate to ensure that organ or body cavity distention does not occur. Passive venting of cryospray may also be achieved in the absence of an active or passive tube or working channel by managing the body lumen to maintain proper circulation and egress of gases. For example, the respective entry point (e.g., esophagus, rectum, etc.) of the body lumen may be maintained in an open configuration to ensure that internal air pressure at or near the site of the cryotherapy procedure remains equal to the atmospheric pressure (e.g., the pressure outside the body). In addition, or alternatively, the position of the patient on the operating table may adjusted (e.g., lying flat, prone, inclined, declined, on their left or right side) to prevent the lumen from partially or completely collapsing under the patient's own weight.

The detachable expandable member 410 may provide distinct advantages over conventional cryotherapy systems which require the expandable member to remain tethered to the delivery device. For example, the ability to remove the delivery device 450 from the body lumen during the cryotherapy procedure eliminates blocking or shielding effects resulting from the position of the delivery device 450 that may prevent the cryospray from contacting the full surface of the treatment area. Additionally, the fixed location of the expandable member allows the medical professional to treat multiple target locations within the body lumen without moving or repositioning the expandable member. The combined benefits of uniform cryospray distribution and the ability to reposition the cryogen delivery catheter may be especially beneficial for treating Barrett's esophagus, which often include unhealthy tissue lesions that are centimeters (or more) in length and cover the full circumference of the esophagus wall.

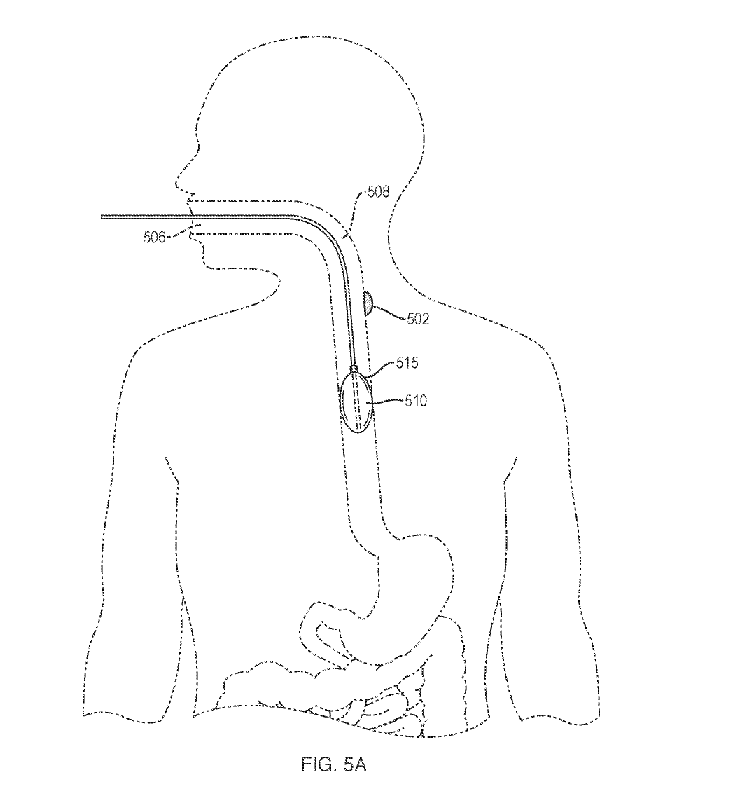

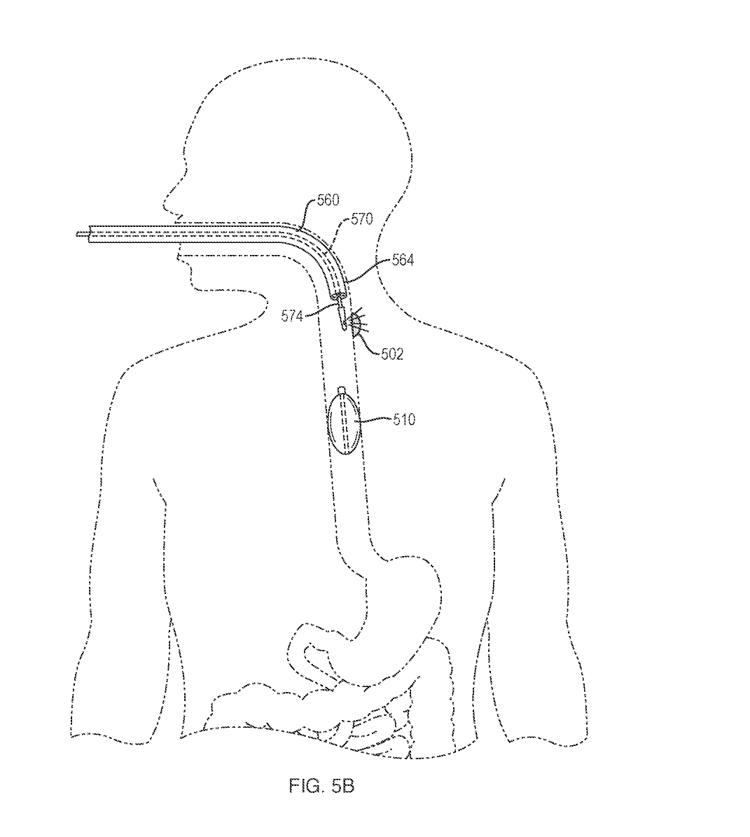

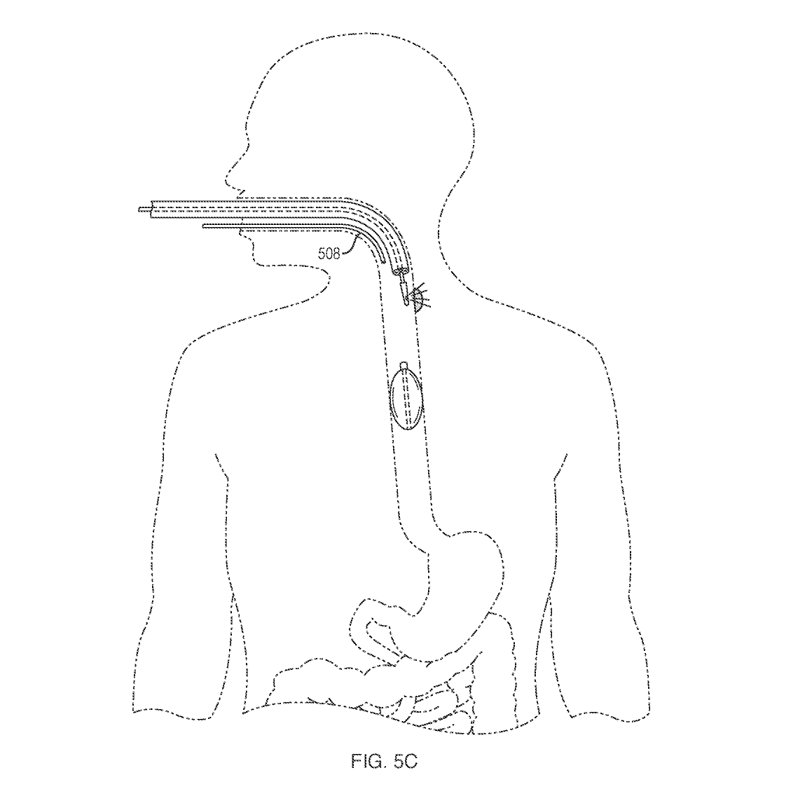

Referring to FIG. 5A, the expandable member 510 may be introduced in the unexpanded configuration through the mouth 506 into the esophagus 508 and distally beyond a target tissue 502. Once properly positioned within the esophagus 508, the expandable member 510 is moved to the expanded configuration such that at least a portion of the outer surface 515 contacts all, or substantially all, of the tissues about a circumference of the esophageal wall. Referring to FIG. 5B, after the delivery device has been detached from the expandable member 510 and withdrawn from the patient, an endoscope 560 is positioned within a portion of the esophagus 508 adjacent to, or in the vicinity of, the target tissue 502. Alternatively, with this and other procedures, the endoscope may be maintained at the target site to visualize detachment and re-acquisition of the expandable member. The delivery catheter may be inserted alongside and outside of the endoscope or may be inserted through a working channel internal to the endoscope. A cryogen delivery catheter 570 is then advanced distally beyond the distal portion 564 of the endoscope 560 such that the distal end 574 is adjacent to the target tissue 502. Cryospray is then delivered to the target tissue as discussed above. Referring to FIG. 5C, in addition, or alternatively, a vent tube 580 may be advanced through the esophagus alongside the endoscope to further assist in evacuation of the cryospray, and other undesirable fluids and materials.

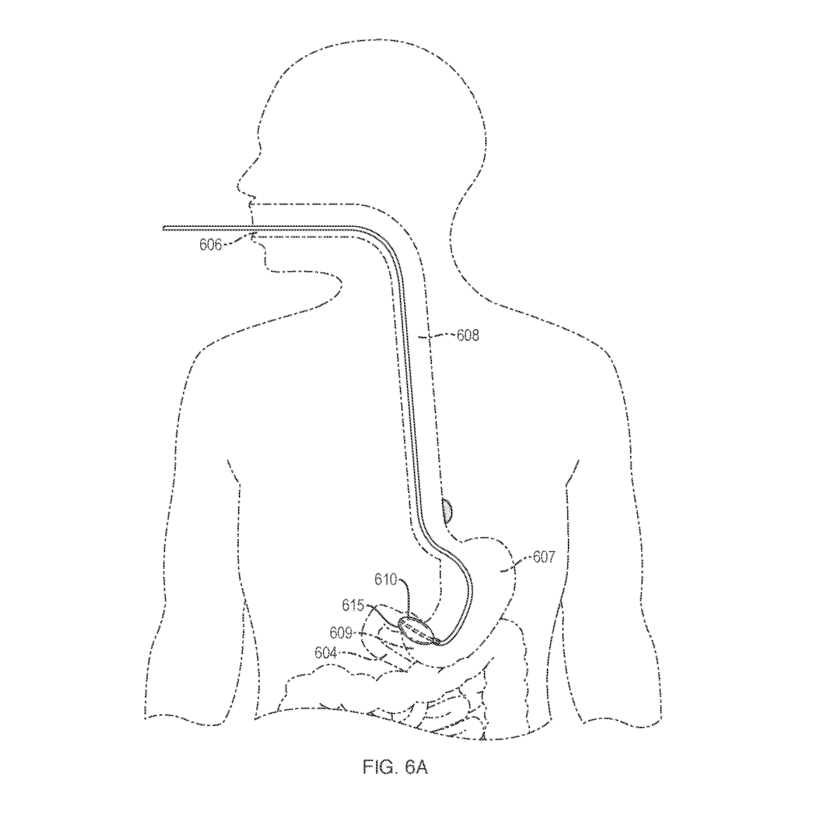

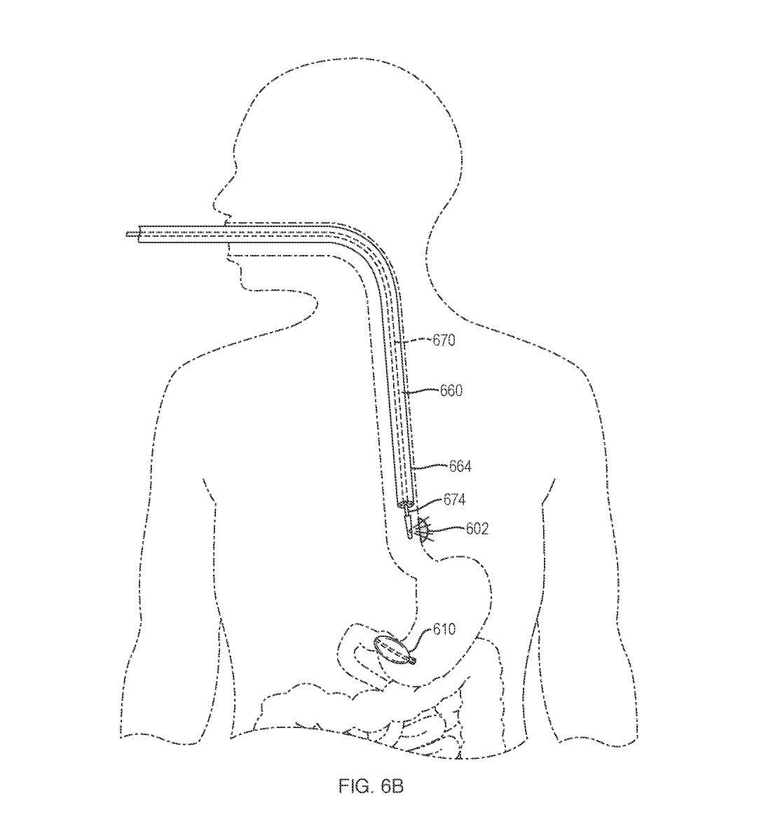

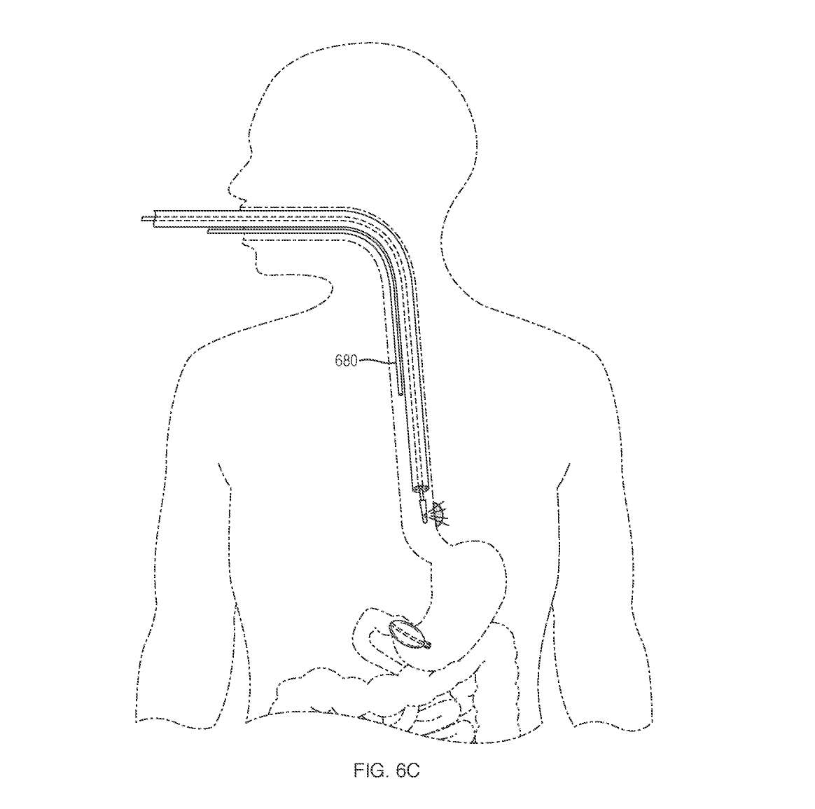

Referring to FIG. 6A, the expandable member 610 may be introduced in the unexpanded configuration through the mouth 606 and positioned within a distal region of the stomach 607 near the pylorus 609 to prevent or significantly inhibit cryospray from entering the duodenum 604. Once properly positioned, the expandable member 610 is moved to the expanded configuration such that at least a portion of the outer surface 615 contacts all, or substantially all, of the tissues about a circumference of the pylorus wall. Referring to FIG. 6B, after the delivery device has been detached from the expandable member 610 and withdrawn from the patient, an endoscope 660 is positioned at, or beyond, the gastroesophageal junction (GEJ) adjacent to the target tissue 602. A cryogen delivery catheter 670 is then advanced distally beyond the distal portion 664 of the endoscope 660 such that the distal end 674 is adjacent to the target tissue 602. Cryospray is then delivered to the target tissue as discussed above. Referring to FIG. 6C, in addition, or alternatively, a vent tube 680 may be advanced through the esophagus to further assist in evacuation of the cryospray, and other undesirable fluids and materials. It is noted that the target tissue 602 may be located anywhere within the esophagus 608, stomach 607, or near the GEJ.

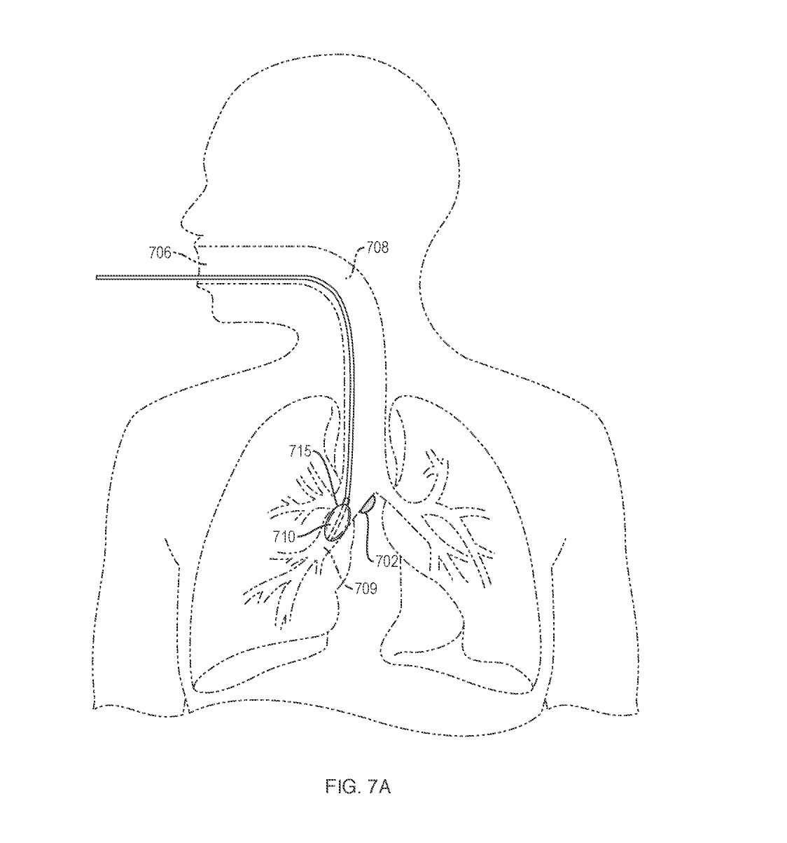

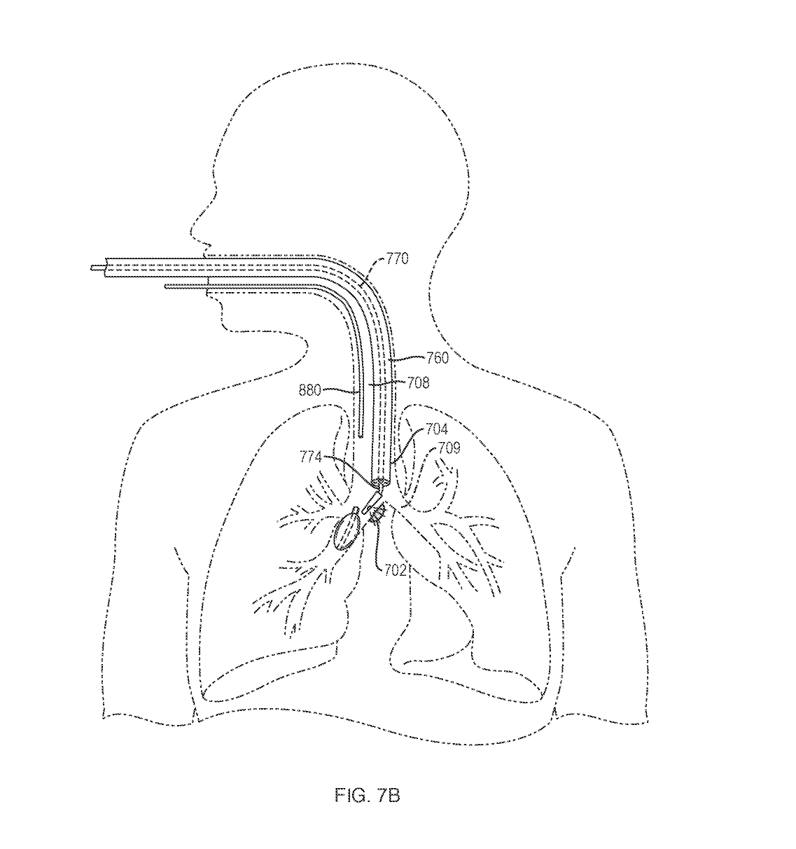

Referring to FIG. 7A, the expandable member 710 may be introduced in the unexpanded configuration through the mouth 706 into the trachea 708 and distally beyond a target tissue 702. Once properly positioned within a bronchial tube 709, the expandable member 710 is moved to the expanded configuration such that at least a portion of the outer surface 715 contacts all, or substantially all, of the tissues about a circumference of the bronchial tube. Referring to FIG. 7B, after the delivery device has been detached from the expandable member 710 and withdrawn from the patient, an endoscope 760 is positioned within a portion of the trachea 708 or bronchial tube 709 adjacent to, or in the vicinity of, the target tissue 702. A cryogen delivery catheter 770 is then advanced distally beyond the distal portion 764 of the endoscope 760 such that the distal end 774 is adjacent to the target tissue 702. Cryospray is then delivered to the target tissue as discussed above. A vent tube 880 may be advanced through the trachea to further assist in evacuation of the cryospray, and other undesirable fluids and materials.

In any of the embodiments described herein, more than one expandable member may be positioned and detached within the target body lumen. Individual delivery devices may be “preloaded” with expandable members, or delivery devices may be reusable and removed from the patient and re-loaded to position multiple expandable members within different body lumens. For example, two or more expandable members may be positioned in the same body lumen as a back-up or redundant measure to ensure that cryospray does not advance distally into the patient. In addition, or alternatively, two or more expandable members may be positioned within separate body lumens (e.g., bifurcated passages or branches) in the vicinity of the target body lumen. By way of non-limiting example, the cryotherapy procedure of FIGS. 7A-7B may further include an expandable member positioned within the bronchial passage of the non-treated lung to prevent or significantly inhibit distal progression of partially or poorly vented cryospray gases.

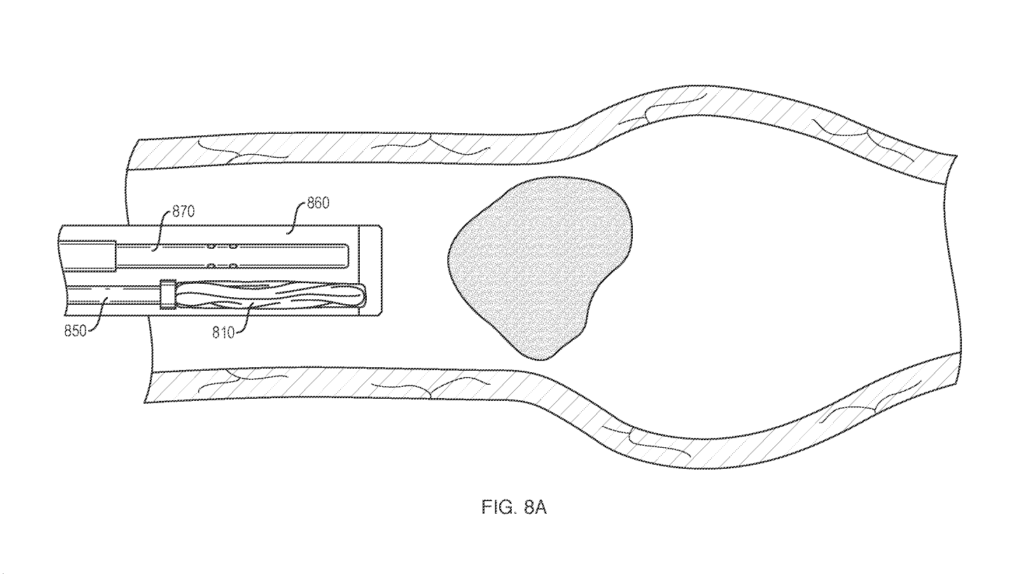

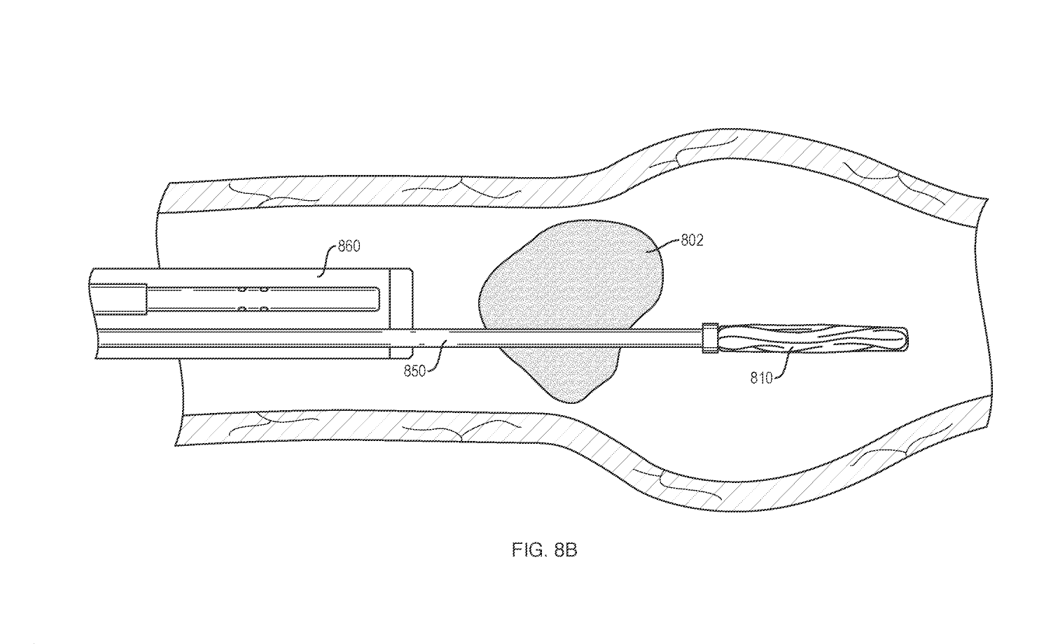

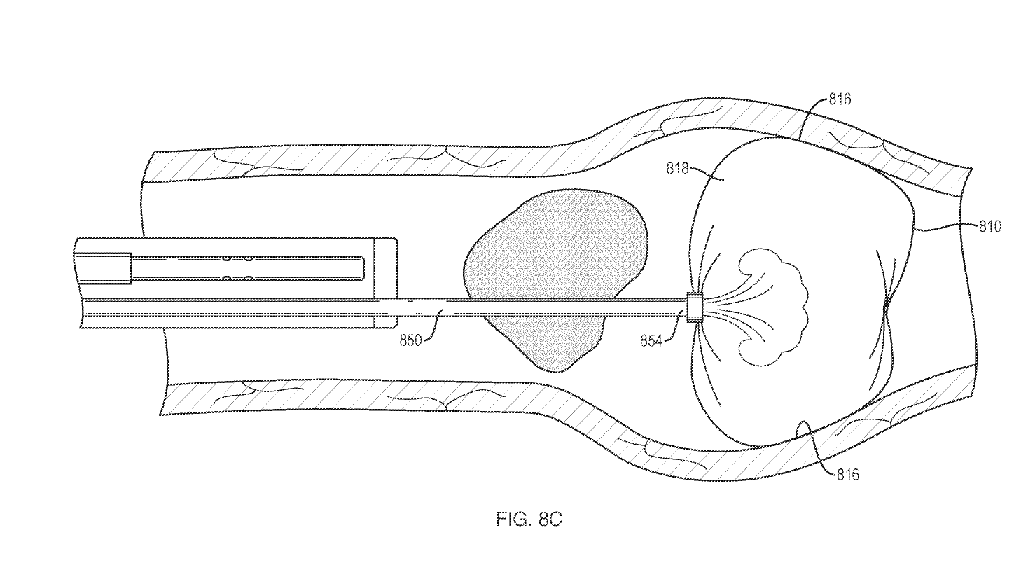

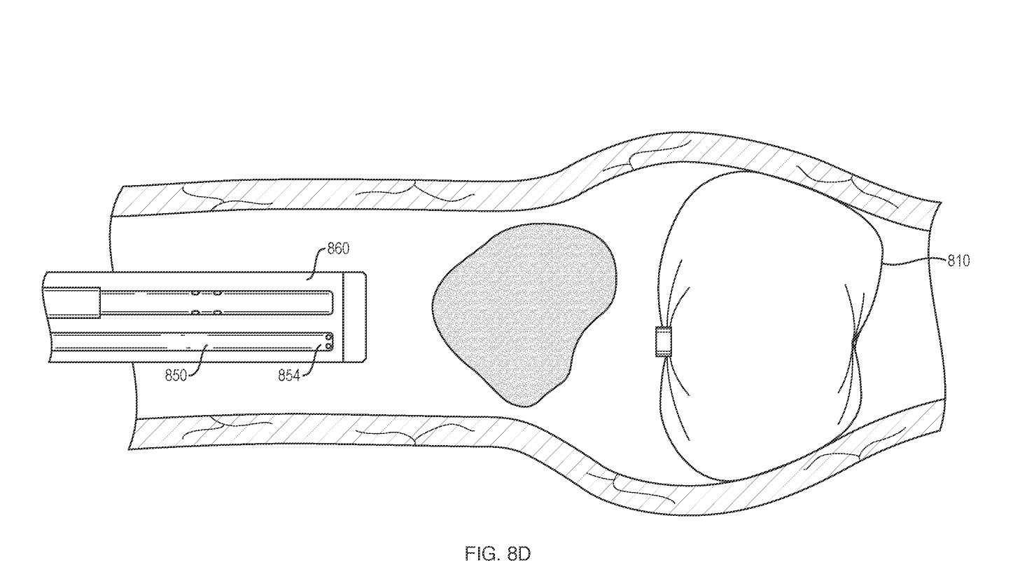

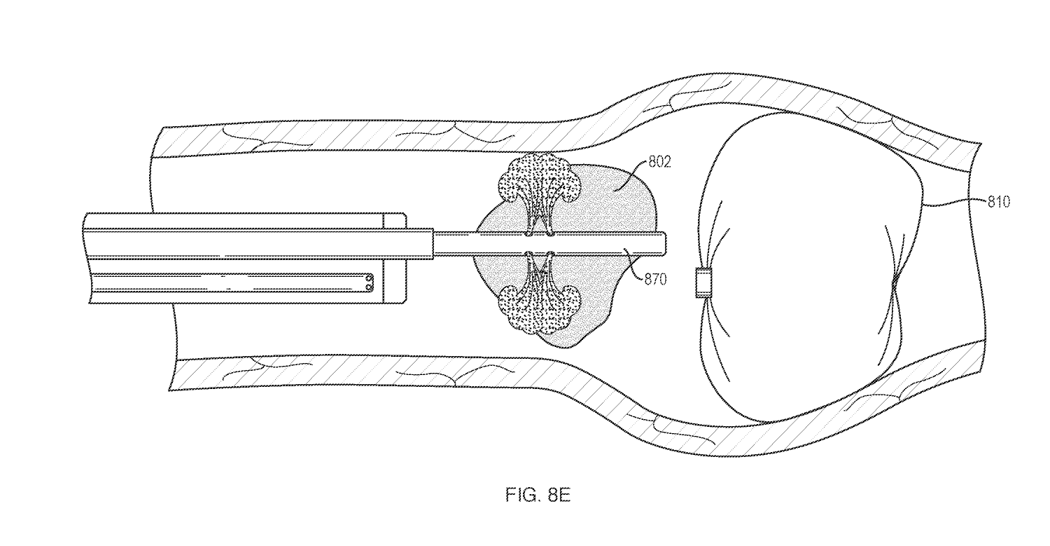

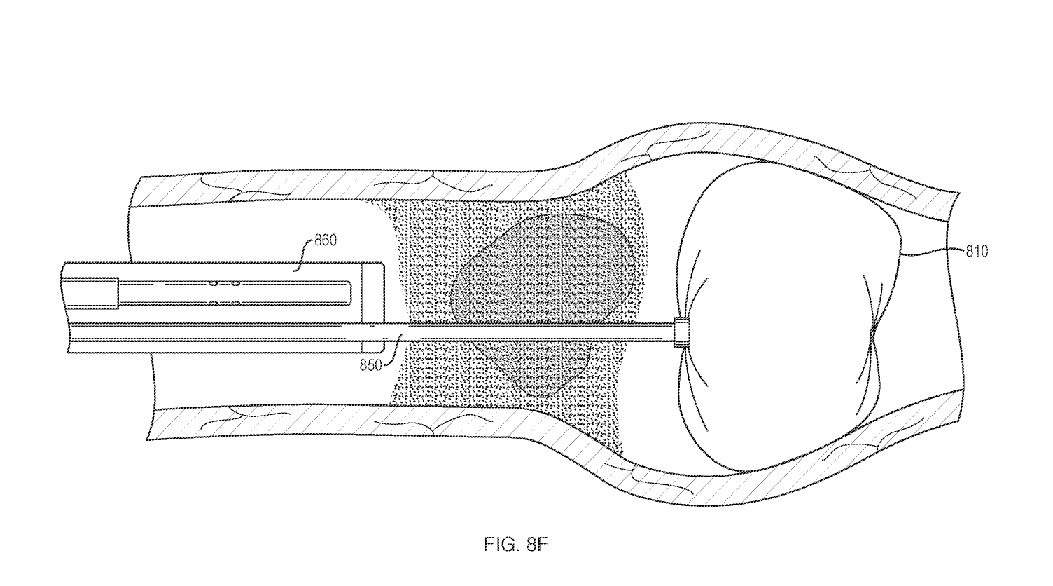

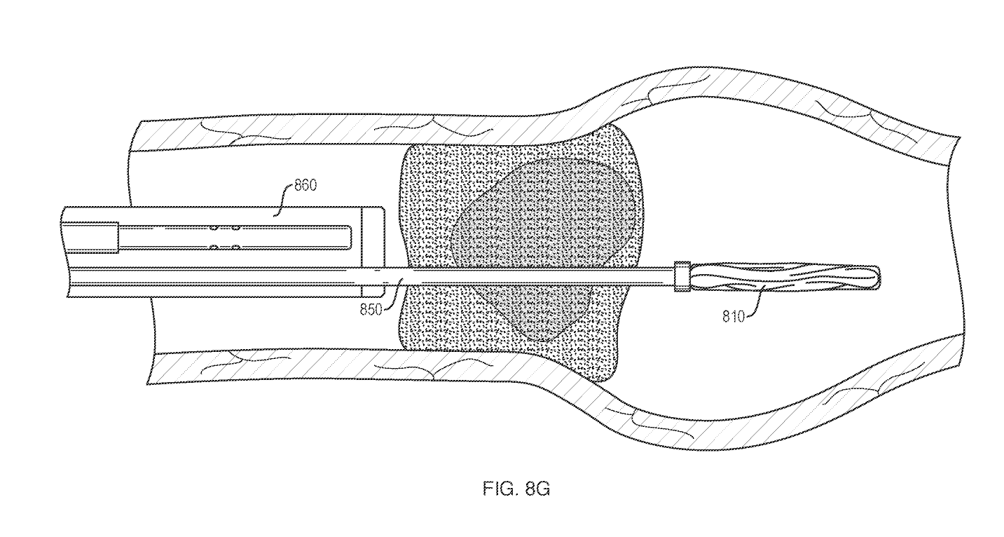

Although the delivery device and detachable expandable member of the present disclosure are depicted throughout the previous figures as being, and in these and other embodiments are capable of delivery, separate from the endoscope, in these and other embodiments, the delivery device and detachable expandable member may be introduced into the patient, in the unexpanded configuration, through a working channel of an endoscope. For example, referring to FIGS. 8A-8G, a detachable expandable member 810 reversibly attached to a delivery device 850, and a cryogen delivery catheter 870, may be advanced into a body lumen within respective working channels of an endoscope 860 (FIG. 8A). The delivery device 850 and detachable expandable member 810 may then be advanced distally beyond a distal end of the endoscope 860 such that the detachable expandable member is positioned distally beyond a target tissue 802 in an unexpanded configuration (FIG. 8B). Inflation fluid may then be delivered through a lumen of the delivery device 850 into an interior region 818 of the detachable expandable member 810 such that an outer surface 816 of the detachable expandable members contacts opposing walls of the body lumen (FIG. 8C). The distal end 854 of the delivery device 850 may then be detached from the detachable expandable member 810 and withdrawn into the working channel of the endoscope 860 (FIG. 8D). The cryogen delivery catheter 870 may then be advanced distally beyond the distal end of the endoscope such that cryogen spray delivered from the cryogen delivery catheter contacts a target tissue 802 proximal to the detachable expandable member 810 (FIG. 8E). When the cryotherapy procedure is complete, the delivery device 850 may be advanced distally beyond the distal end of the endoscope 860 to reestablish a connection with the detachable expandable member 810 (FIG. 8F). The inflation fluid may then be removed from within the interior region of the detachable expandable member 810 through the lumen of the delivery device 850 such that the detachable expandable member returns to the unexpanded configuration. The delivery device 850 and detachable expandable member 810 may then be retracted into the working channel of the endoscope 860 (FIG. 8G), and the endoscope with the delivery device and the cryogen delivery catheter removed from the patient.

Alternatively, the delivery device and detachable expandable member may be slidably attached to an outer surface of the endoscope using one or more clips or fastening elements, as are known in the art. For example, the endoscope may include a detachable external lumen or rail system through which the delivery device may be slidably disposed.

Any of the embodiments described herein may further benefit from passive or active venting of the treatment area (i.e., proximal to the expandable member) through a working channel of the endoscope and/or a working channel of the cryogen delivery catheter. Passive venting may be further facilitated, independent of such vent tubes and/or working channel(s), by managing the body lumen to maintain proper circulation and egress of gases, as discussed above.

All of the devices and/or methods disclosed and claimed herein can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

What is claimed is:

1. A system, comprising:
a delivery device, comprising:
a distal end;
a proximal end; and
a lumen extending therebetween;
an expandable member, comprising:
an outer surface;
an inner surface defining an interior region; and
a valve disposed on the outer surface of the expandable member, and in fluid communication with the interior region;
wherein the distal end of the delivery device is reversibly attachable to the valve such that the interior region of the expandable member is in fluid communication with the lumen of the delivery device via the valve.

2. The system of claim 1, wherein the distal end of the delivery device includes an attachment element configured to engage a corresponding attachment element of the valve.

3. The system of claim 2, wherein the distal end of the delivery device includes a male attachment element configured to engage a corresponding female attachment element of the valve.

4. The system of claim 3, wherein the corresponding male and female attachment elements include threaded surfaces.

5. The system of claim 3, wherein the corresponding male and female attachment elements include luer lock surfaces.

6. The system of claim 3, wherein a portion of the male attachment element is configured to expand within a portion of the female attachment element.

7. The system of claim 1, wherein the valve is seated within a housing having a funnel member configured to guide the distal end of the delivery device into the housing.

8. A system, comprising:
a delivery device having a distal end, a proximal end and a lumen extending therebetween;
an expandable member defining an interior region; and
a valve disposed between and having a fluid communication to the lumen and the interior region,
wherein the valve and expandable member are reversibly detachable from the distal end of the delivery device.

9. The system of claim 8, wherein the distal end of the delivery device includes a male attachment element configured to engage a corresponding female attachment element of the valve.

10. The system of claim 9, wherein the male attachment element is configured to expand when inserted a predetermined depth into the female attachment element.

11. The system of claim 8, further comprising a support member extending along a longitudinal axis of the expandable member within the interior region.

12. The system of claim 8, wherein the expandable member is a balloon and comprises a non-compliant or semi-compliant polymer material selected from the group consisting of PEBAX, PET, PEN, PBT, PEEK, Hytrel, polyurethane and nylon.

13. The system of claim 8, further comprising an endoscope, wherein the endoscope includes a detachable external lumen or rail system.

14. The system of claim 8, wherein the system is slidably disposed within a working channel of an endoscope.

15. The system of claim 8, wherein the system includes one or more fastening elements configured to slidably attach to an outer surface of an endoscope.

16. A method, comprising:
positioning a delivery device that includes a detachable expandable member at a target location within a body lumen;
inflating the detachable expandable member to contact opposing walls of the body lumen at the target location;
detaching the detachable expandable member from the delivery device; and
performing a procedure in the body lumen at a position proximal to the target location.

17. The method of claim 16, further comprising re-attaching the delivery device to the detachable expandable member.

18. The method of claim 17, further comprising deflating the detachable expandable member.

19. The method of claim 18, further comprising removing the delivery device and detachable expandable member from the body lumen.

20. The method of claim 16, wherein the procedure is a cryotherapy procedure.
